(19) United States
(12) Patent Application Publication
(10) Pub. No.: US 2016/0367714 A1
(43) Pub. Date: Dec. 22, 2016

(54) PLASTIC COMPONENT FOR DELIVERY OF FRAGRANCE

(71) Applicant: Henkel AG & Co. KGaA, Duesseldorf (DE)

(72) Inventors: Salvatore Fileccia, Oberhausen (DE); Lars Lieberwirth, Shanghai-Pudong (CN)

(73) Assignee: Henkel AG & Co. KGaA, Duesseldorf (DE)

(21) Appl. No.: 15/257,676

(22) Filed: Sep. 6, 2016

Related U.S. Application Data
(63) Continuation of application No. PCT/EP2015/056581, filed on Mar. 26, 2015.

(30) Foreign Application Priority Data
Mar. 31, 2014 (EP) 14162961.8

(51) Int. Cl. A61L 9/04; C08K 11/00; B65D 25/20; C11B 9/00

(52) U.S. Cl. CPC A61L 9/042; C11B 9/0003; C08K 11/00; B65D 25/20

(57) ABSTRACT

Plastic component 1 for releasing fragrance, comprising at least a first wall 2 with a first side 4 and a second side 5 characterized in that, a second wall 3 is formed at least partially in parallel with the first wall 2 wherein the first wall 2 is spaced apart from the second wall 3 by at least two partition walls.

### FIELD OF THE INVENTION

The present invention generally relates to a plastic component for delivery of fragrance to the surrounding ambient.

### BACKGROUND OF THE INVENTION

Accurate dispensing of washing and/or cleaning agents is increasingly having a higher significance. Although the substances for domestic use are being produced in larger quantity, they are not necessarily getting cheaper. The cleaning agents are being increasingly engineered and tailored to satisfy the user needs, which translates into an overall higher efficiency and lower volumes of agent needed. Further, there is a need optimize and finally reduce the amounts of released substances into the environment, reducing the environmental impact. To achieve these goals, the washing and/or cleaning agents should be dispensed in the correct amount, position, and at the correct time.

For dispensing fragrance in the ambient it is known to have fragrance in a plastic so that the plastic delivers the fragrance to the ambient during time. Suitable formulations for injection moulding scented plastics are known from the document WO 2007/133438 A2. This document discloses the production a scented plastic part to be hold in position in a cap assembly of a bottle.

Conventional fragrance plastic components change shape during time, because the delivery of fragrance to the ambient implies in a loss of volume. Thus it is difficult to keep these components in place, further it may cause an unpleasant view depending on the form and magnitude of the distortion.

The problem of the invention is thus to provide a plastic component for fragrance delivery which is stable upon loss of volume.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

Plastic component (1) for releasing fragrance, comprising at least a first wall (2) with a first side (4) and a second side (5) characterized in that, a second wall (3) is provided at least partially in parallel with the first wall (2) wherein the first wall (2) is spaced apart from the second wall (3) by at least two partition walls (6).

### BRIEF DESCRIPTION OF THE DRAWINGS

The present invention will hereinafter be described in conjunction with the following drawing figures, wherein like numerals denote like elements, and

FIG. 1A shows a plastic component (1) for releasing fragrance;

FIG. 1B is a cross section showing that the space between the walls are formed by non-parallel wall-sides (17); and

FIG. 2 shows a suitable holder can be part of a container (18).

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The invention concerns a plastic component for releasing fragrance, comprising at least a first wall with a first side and a second side. A second wall is provided at least partially in parallel, preferentially in parallel, with the first wall. The first wall is spaced apart from the second wall by at least two partition walls. The fragrance is comprised in the material of the plastic component, preferably at least in the material of the first and second walls.

Conventional fragrance plastic elements change shape during time, because the delivery of fragrance to the ambient implies in a loss of volume. It was found that this may cause issues, especially for strip like plastic components, the change in shape may cause distortions which may be so dramatic that it may no longer fit into an appropriate fitting place. Or the component may even fall out of its fitting place.

Due to the double wall construction and the partition walls the change of shape is compensated so that the distortion is minimized. Firstly, because one of the walls supports the other, and secondly because the walls lose volume at the same time, at similar rates. With this arrangement it is even possible to have surfaces to be slightly different at the first and second walls, while still providing the compensation effect.

The walls are separated apart by at least two partition walls. An increased number of partition walls can be envisaged so that the component can support a stronger mechanical strain, including mechanical deformation strain.

In one development of the invention, the area of the first side is larger than the planar area delimited by its outer contour. In one further development of the invention, the plastic component has an, at least partially, structured surface. The structured surface increases the area of the first side. The increased area is favourable if a stronger rate of fragrance delivery to the ambient is envisaged. It was surprisingly found that the increased surface area (structured or not) also improves the stability against deformation from volume loss of the component. The first wall having an overall larger area, does lose volume much faster than the second wall, this leads to a stronger tendency of deformation. However, due to the structured surface, the first wall itself is mechanically more flexible and is kept in its shape by the second wall and the partition walls. The above effect is stronger for strip like walls if the structured surface is wave-like, where valleys and peaks are repeated in the direction of the length of the strip. The wave-like shape can be in principle of any shape such as sinusoidal, triangular, rectangular, or combinations thereof.

For appropriate fastening of the plastic component, the second wall may comprise a fastening means for attaching the component to a suitable holder. The fastening means and its suitable holder preferably forms a force-locked connection. In case of strip like walls, it is preferred that the component comprises two fastening, both placed apart from each other so that most or all of the walls is provided between the fastening means. This further helps to increase mechanical stability, and stability against deformation by loss of volume. As an example the fastening means may be provided at or close to the ends of the walls.

It is further preferred that each of the fastening means is connected to the second wall. Further, it is preferred that each of the fastening means is connected to the second wall via a connector, further preferable a curved connector, which acts as a spring if under mechanical tension.

The fastening means itself, is preferably made of the same material as the walls, thus it contributes to the delivery of fragrance to the ambient.

For all embodiments of the invention, each fastening means can comprise at least two wall segments, which can be elastically deformed when inserting in a suitable holder, thus providing a force-locked connection. Examples if such segments are selected from U-Form or V-Form. The vertex of the two wall segments are preferably connected to the connector. The wall segments may be part of an open structure, thus having ends. The wall segments may also be part of a closed structure, thus being part of the same wall which may itself have a completely closed cross section, or comprise openings, such as a circular cross-section.

The space in between the wall segments, if existing, may be used to store additional fragrance delivery material. For instance a fragrance in a gel matrix, or a chamber comprising a fragrance composition and a semi-permeable membrane. The additional fragrance is preferably different from the fragrance of the material of the plastic component. With this additional fragrance it is possible to fine tune the scent experience for the consumer. This is much easier than having the delivery of a fine tuned mixed fragrance out of the plastic component itself (e.g. plastic walls).

The invention also envisages that the plastic component preferably comprises demoulding structures on at least one of the second wall or the second side of the first wall, preferably on both. The cross-section along the openings, from one side to the other, formed from the space between the first wall and the second wall, is preferably non-planar, more preferably cone-shaped. The shape of the cross-section is such that a demoulding, from the form in which the component is moulded, is facilitated. This was found to decrease the defect rate in production, especially for injection-moulded components which may be defected when using force on a demoulding step.

It is preferred that the first wall is spaced apart from the second wall by a minimum distance of between 1 mm and 5 mm.

The plastic component is preferably made of a material comprising at least one of HDPE, PP, TPE, PS, PA, EVA.

The plastic component comprises at least 15 weight % of fragrance composition. Typical fragrance scents are lemon, pine, orange, eucalyptus, ocean blue, hygiene, lime, grape or a combination thereof.

The invention will be further described with the following Figures.

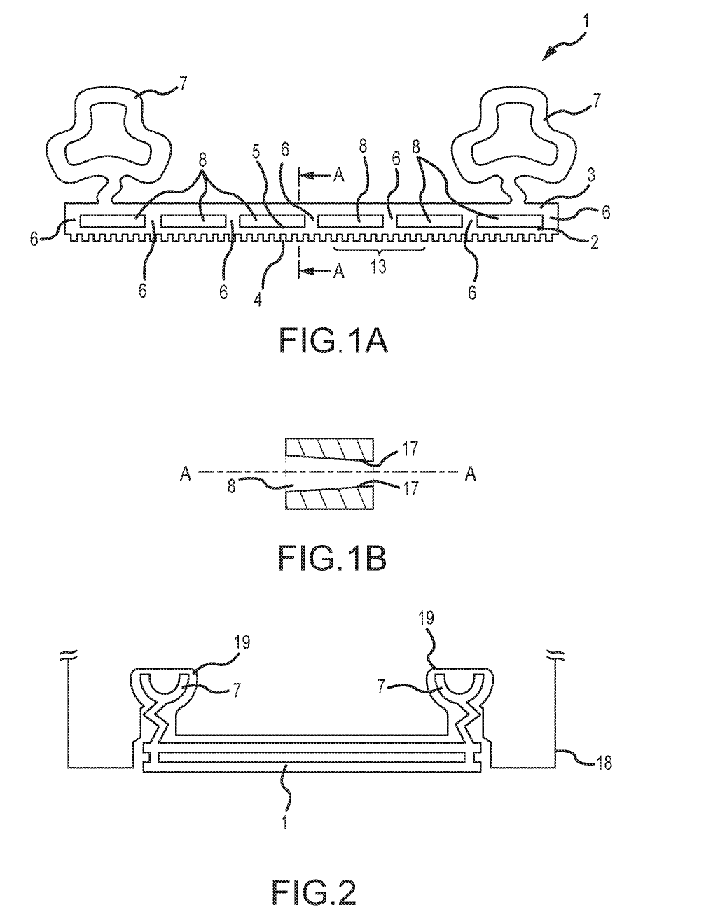

FIG. 1 shows a plastic component (1) for releasing fragrance, comprising at least a first wall (2) with a first side (4) and a second side (5). A second wall (3) is provided in parallel with the first wall (2). The first wall (2) is spaced apart from the second wall (3) by at least two partition walls (6), in the present figure by seven partition walls (6).

In this embodiment of the invention, the plastic component has a structured surface (13) on the first side (4). The sign (13) is meant to point at the whole structured part, nonetheless the structured part can be only on part of the first side (4), but is preferably the whole first side (4). The structured surface (13) increases the area of the first side (4).

The spaces between the walls 8 further contribute to the delivery of fragrance. The cross-section A-A, shown in FIG. 1B (not in scale), shows that the space between the walls are formed by non-parallel wall-sides (17), this is a preferred execution of the invention due to the easier demoulding from a form.

The fastening means (7) are shaped such that they can be elastically deformed and attached to a suitable holder (19), as in FIG. 2. The suitable holder (19), as in FIG. 2, can be part of a container (18).

### REFERENCE NUMBERS

1 Plastic component
2 first wall
3 second wall
4 first side
5 second side
6 partition walls
7 fastening means
8 space between walls, demoulding structure
9 distance
11 area of the first side
12 outer contour
13 structured surface
17 non-parallel wall sides
18 container
19 suitable holder

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A plastic component (1) for releasing fragrance, comprising at least a first wall (2) with a first side (4) and a second side (5) wherein a second wall (3) is provided at least partially in parallel with the first wall (2) wherein the first wall (2) is spaced apart from the second wall (3) by at least two partition walls (6).

2. The plastic component (1) according to claim 1, wherein the second wall (3) comprises a component for fastening (7) for attaching the component to a suitable holder.

3. The plastic component (1) according to claim 1, wherein at least one of the second wall (3) or the second side (5) of the first wall (2) comprises demoulding structures (8).

4. The plastic component (1) according to claim 1, wherein the first wall (2) is spaced apart from the second wall (3) by a minimum distance (9) of between 1 mm and 5 mm.

5. The plastic component (1) according to claim 1, wherein the area of the first side (11) (4) is larger than the planar area delimited by its outer contour.

6. The plastic component (1) according to claim 1, wherein the first side (4) has an, at least partially, structured surface (13) for increasing the area of the first side.

7. The plastic component (1) according to claim 1, wherein the plastic component (1) is composed of a material (14) comprising at least one of HDPE, PP, TPE, PS, PA, EVA.

8. The plastic component (1) according to claim 1, wherein the material (14) comprises at least 15 weight % of fragrance composition lemon, pine, orange, eucalyptus, ocean blue, hygiene, lime, grape or a combination thereof.
